(19) United States
(12) Patent Application Publication
(10) Pub. No.: US 2018/0064542 A1
(43) Pub. Date: Mar. 8, 2018

(54) ARTIFICIAL HIP JOINT IMPLANT

(71) Applicant: Klaus G. Knöchel, Ulm (DE)

(72) Inventor: Klaus G. Knöchel, Ulm (DE)

(21) Appl. No.: 15/562,066

(22) PCT Filed: Jun. 5, 2015
(86) PCT No.: PCT/DE2015/100224

§ 371 Date: Sep. 27, 2017

(30) Foreign Application Priority Data
Mar. 27, 2015 (DE) 10 2015 104 798.5

(51) Int. Cl. A61F 2/36

(52) U.S. Cl. CPC A61F 2/3609; A61F 2002/3055; A61F 2220/0025; A61F 2/3662

(57) ABSTRACT

The invention relates to an artificial hip joint implant (1), having a hip head (2), a femur neck (3) and a femur shaft (4) connected thereto, wherein the femur shaft (4) has an upper part (41) facing the shaft attachment (3) and a lower part (42), and the upper part (41) is connected to the lower part (42) by a length-adjusting device (51, 52, 6).

The present invention relates to an artificial hip joint implant, having a hip head, a femur neck and a femur shaft connected thereto. Furthermore, the invention also relates to a method for adjusting the length of such a hip joint implant.

As a result of the use of artificial hip joint implants, there are often differences in leg length and skewed positions of the pelvis due to inaccuracies and boundary conditions occurring during surgery, such that it may even be necessary in particularly serious cases for the patient to undergo a repeat surgery in order to be able to walk without pain.

Numerous forms of artificial hip joint implants having different femur shafts are known in the state of the art, such that the choice of the proper femur shaft is made on the basis of the length and dimensions.

The object of the present invention is to make available an improved hip joint implant.

This object is achieved by an artificial hip joint implant according to the features of claim 1 and a method for adjusting the length of an artificial hip joint implant according to the features of claim 8.

It is provided according to the invention that the femur shaft has an upper part facing the shaft attachment and a lower part, and the upper part is connected to the lower part by a device for adjusting the length.

The present invention thus relates to a hip joint implant, such that the length of the femur shaft can be adjusted postoperatively. This adjustment can be carried out in any orthopedic practice and can also be repeated several times in order to achieve a horizontal hip axis.

Differences in leg length and skewed positions of the pelvis, which occur frequently after implantation of an artificial hip joint, can thus be eliminated without having to subject the patient to a repeat surgery.

Furthermore, a higher quality of movement is achieved in this way, the patient has an improved walking pattern, and there is a definite reduction in the consequences of wear and material fatigue thanks to this balanced symmetry.

According to one advantageous embodiment of the invention, a thread designed to run in the opposite direction from the other thread is provided in the upper part and the lower part of the femur shaft, respectively, and a threaded rod, which is provided with corresponding threads in opposite directions accordingly is guided in both threads, connecting the upper part to the lower part of the femur shaft.

A particularly preferred embodiment of the invention provides that a magnet is provided in or on the threaded rod. This magnet can enable rotation of the threaded rod in one direction or the other by means of a variable magnetic field, so that the distance between the upper part and the lower part of the femur shaft can be defined in a variable manner.

It is advantageously provided that the magnet is disposed in a cavity in the upper or lower part of the femur shaft and is mechanically in form-fitting or force-locking engagement with the threaded rod.

The upper part or the lower part of the femur shaft is preferably guided in a sleeve in the opposite part.

The sleeve is advantageously designed so that it seals the part of the femur shaft guided therein.

According to an advantageous embodiment of the invention, hollow ground interiors protruding are provided in or on the femur shaft, tapering in the direction toward the lower part of the femur shaft. This improves the attachment of the bone to the hip joint implant.

According to another aspect of the invention, a method for adjusting the length of an artificial hip joint implant according to any one of claims 3 to 7 is proposed and is characterized in that the magnet is acted upon by a magnetic field in such a way that the magnet is induced to rotate, and therefore a movement of the upper part and the lower part of the femur shaft toward or away from one another is induced by means of the threaded rod, which is mechanically coupled to the magnet.

This requires an external remote control, which is placed on the hip, and a gear is moved by rotation of an adjusting magnet that is integrated into the implant. This gear lengthens or shortens the leg until the two legs have been balanced according to a measuring bar.

Additional advantageous embodiments are derived from the additional dependent claims or possible sub-combinations thereof.

The invention is explained in greater detail below on the basis of the drawings. The schematic diagrams show in detail:

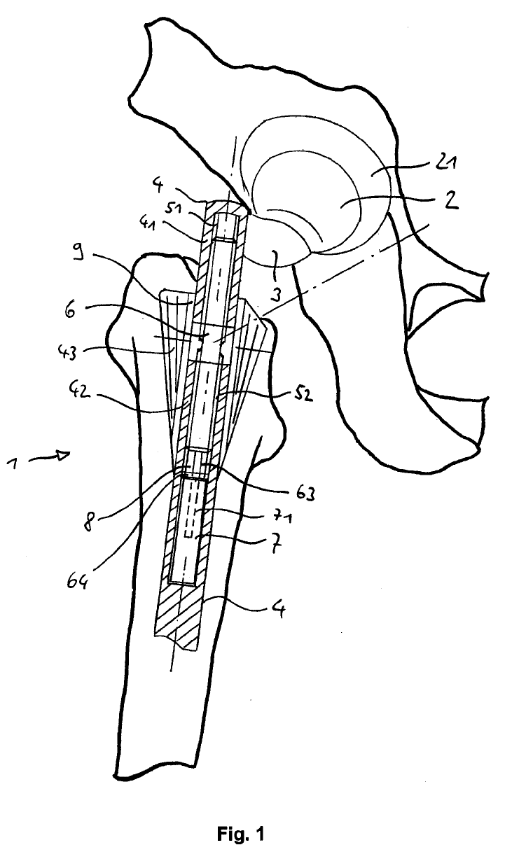

FIG. 1 a schematic sectional diagram through a hip joint implant according to the invention, shown schematically after insertion into the bone,

FIG. 2 a schematic view of the hip joint implant from FIG. 1,

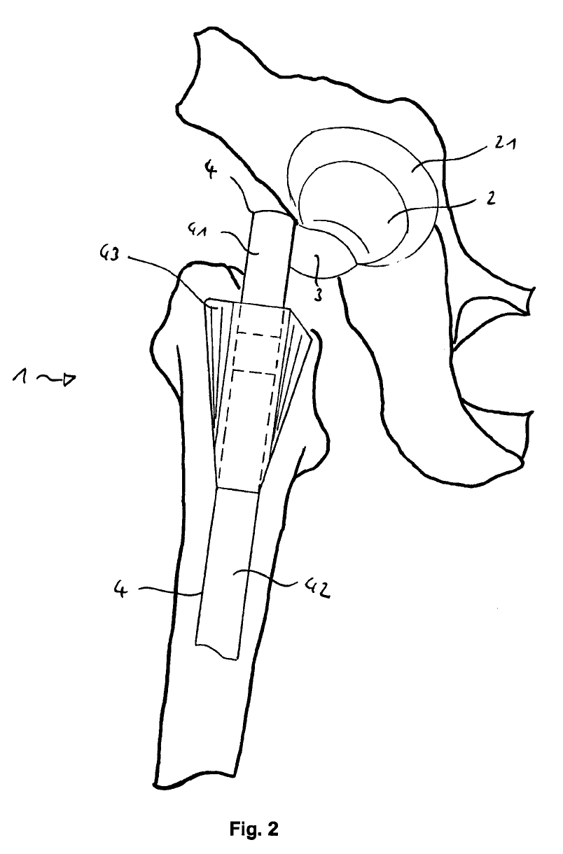

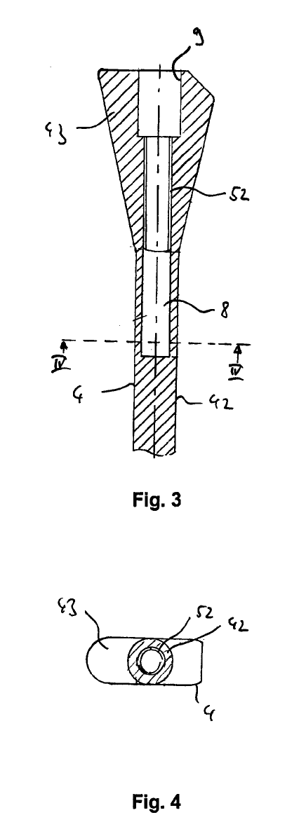

FIG. 3 a schematic detailed diagram of the lower part of the femur shaft in a sectional diagram,

FIG. 4 a schematic sectional diagram of the lower part of the femur shaft from FIG. 3, as seen in the direction IV,

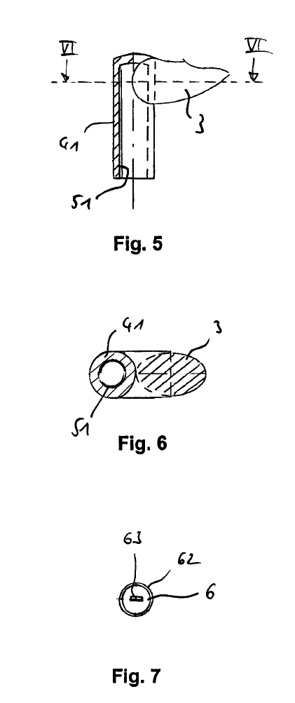

FIG. 5 a schematic detail diagram of the upper part of the femur shaft in a sectional diagram,

FIG. 6 a schematic sectional diagram of the upper part of the femur shaft from FIG. 5, as seen in the direction VI,

FIG. 7 a schematic diagram of the threaded rod as seen in the direction of the axis of rotation,

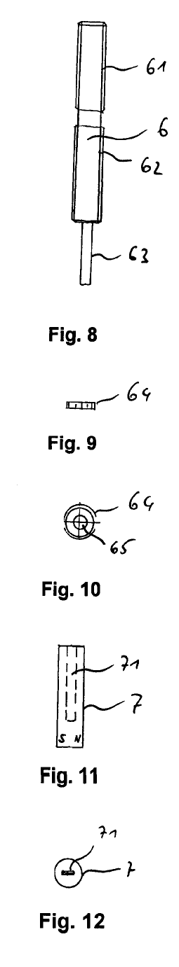

FIG. 8 a schematic view of the threaded rod from the side,

FIG. 9 a detailed diagram of a threaded ring as seen from the side,

FIG. 10 a top view of the threaded ring from FIG. 9,

FIG. 11 a schematic view of the magnetic as seen from the side, and

FIG. 12 a schematic diagram of the magnet, as seen in the direction of the axis of rotation.

The same reference numerals in the figures denote the same elements or those having the same effect.

FIG. 1 shows an artificial hip joint implant 1, which is in a bone in its transplanted position and has a head 2 of the hip, supported in a hip socket 21, a femur neck 3 and a femur shaft 4 connected thereto.

According to the invention, the femur shaft 4 has an upper part 41 facing the shaft attachment 3 and the lower part 42, while the upper part 41 is connected to the lower part 42 by means of a length-adjusting device 51, 52, 6.

In the upper part 41 and the lower part 42 of the femur shaft, the longitudinal adjustment device is formed by a thread 51, 52, each of which is designed to run opposite the other, and a threaded rod 6 provided with corresponding opposing threads 61, 62 is guided in both threads 51, 52, which connect the upper part 41 and the lower part 42 of the femur shaft 4 to one another.

To drive the threaded rod without surgical insertion through the tissue from the outside, a magnet 7, which engages with the threaded rod 6, is provided. The magnet can be induced to rotate by an alternating magnetic field that is acted upon by an externally generated alternating magnetic field, so that the magnet shortens or lengthens the distance between the upper part 41 and the lower part 42 of the femur shaft according to the opposing threads.

To do so, the magnet 7 is arranged in a cavity 8 in the lower part 42 of the femur shaft 4 and is mechanically engaged in a form-fitting manner with a corresponding protrusion receptacle 72 by means of an engagement protrusion 63 on the threaded rod 6. In this regard, see also the detailed descriptions of FIGS. 7 through 12. In the cavity 8, the movement of the magnet 7 is limited toward the top by a threaded ring 64.

In order to ensure a good seal with respect to the penetration of tissue or body fluids, the upper part 41 of the femur shaft 4 is guided in a sleeve 9 in the opposing lower part 42, such that the sleeve 9 closes the part of the femur shaft 4 forming a seal with respect to that part.

In addition, the exemplary embodiment shows that hollow ground interiors 43 protruding on the femur shaft 4 are molded in or on the femur shaft, tapering in the direction of the lower part 42 of the femur shaft 4.

FIG. 2 shows again a schematic view of the hip joint implant from FIG. 1.

FIGS. 3 and 4 show a detailed schematic diagram of the lower part 42 of the femur shaft 4 in a sectional diagram and a schematic sectional diagram of the lower part of the femur shaft 4 from FIG. 3, as seen in the direction of view IV.

FIG. 5 shows a schematic detailed diagram of the upper part of the femur shaft 41 in a sectional diagram and FIG. 6 shows a schematic sectional diagram of the upper part of the femur shaft from FIG. 5 in the direction of view VI.

The threaded rod is shown in FIG. 7 in a schematic diagram in the direction of the axis of rotation. Accordingly, FIG. 8 shows the threaded rod 6 from the side, showing the engagement protrusion 63 arranged thereon.

FIGS. 9 and 10 show the threaded ring 64 bordering the space for the magnet 7. The engagement protrusion 63 of the threaded rod 6 can pass through the small opening 65.

Finally, FIGS. 11 and 12 show views of the magnet 7 as seen in the direction of the axis of rotation and as seen from the side. This also shows schematically an exemplary magnetization direction (N, S) of the magnet 7, to which an external alternating field can be applied.

### LIST OF REFERENCE NUMERALS

1 hip joint implant
2 head of hip
21 hip socket
3 femur neck
4 femur shaft
41 upper part
42 lower part
43 hollow ground interior
51,52 thread
6 threaded rod
61,62 thread
63 engagement protrusion
64 threaded ring
65 opening
7 magnet
71 receptacle for protrusion
8 cavity
9 sleeve

1. An artificial hip joint implant,
having a hip head, a femur neck and a femur shaft connected thereto,
wherein the femur shaft has an upper part facing the shaft attachment and a lower part, and the upper part is connected to the lower part by a length-adjusting device,
wherein a thread is provided in the upper part and in the lower part of the femur shaft, each thread being designed to be opposite the other and a threaded rod provided with corresponding opposing threads is guided in both threads, said threaded rod connecting the upper part and the lower part of the femur shaft to one another.

2. The hip joint implant according to claim 1,
wherein a magnet is provided in or on the threaded rod.

3. The hip joint implant according to claim 2,
wherein the magnet is arranged in a cavity (8) in the upper or lower part of the femur shaft and is mechanically engaged with the threaded rod in a form-fitting or force-locking engagement.

4. The hip joint implant according to claim 1,
wherein the upper part and the lower part of the femur shaft are guided in a sleeve in the opposing part.

5. The hip joint implant according to claim 4,
wherein the sleeve closes the part of the femur shaft with a seal which is guided with respect to the opposite part.

6. The hip joint implant according to claim 1,
wherein hollow ground interiors protruding on the femur shaft and taper in the direction of the lower part of the femur shaft.

7. A method for adjusting the length of an artificial hip joint implant according to claim 2,
wherein the magnet is acted upon by a magnetic field such that the magnet is made to rotate and thereby executes a movement of the upper part and the lower part of the femur shaft toward or away from one another by means of the threaded rod which is mechanically coupled to the magnet.
